(19) United States
(12) Plant Patent Application Publication
(10) Pub. No.: US 2005/0108799 P1
(43) Pub. Date: May 19, 2005

(54) Pond cypress tree named 'Morris'

(50) TAXODIUM ASCENDENS; MORRIS

(75) Inventor: Earl Cully, Jacksonville, IL (US)

Correspondence Address: Earl Cully, 846 Hoagland Road, Jacksonville, IL, 62650, US

(73) Assignee: Heritage Trees, Inc., Jacksonville, IL (US)

(21) Appl. No.: 10/713,534

(22) Filed: Nov. 14, 2003

(51) Int. Cl. A01H 7/00

(52) U.S. Cl. PLT/213

(57) ABSTRACT

A new and distinct pond cypress named ‘Morris’ characterized by its medium oval habit of growth, outstanding foliage, its ability to resist breakage from wind and ice, cold hardiness, and its rapid growth

The present invention comprises a new and distinct cultivar of pond cypress, botanically known as Taxodium ascendens, and referred to by the cultivar name ‘Morris’ (FIG. 1).

The initially discovered tree is growing in a cultivated area on the home grounds of inventor Earl Cully, 846 Hoagland Road, Jacksonville, Ill., in Morgan County, in Township 14, Range 10.

The new cultivar ‘Morris’ is the result of small scion given to me in the mid-1960s. J. C. McDaniel had taken the scion from a pond cypress growing at the Morris Arboretum, located in Philadelphia, Pa. He took this scion with the permission of Dr. John Fogg, a friend of his, who was at that time Director of the Morris Arboretum. (Both of these men are now deceased.)

I grafted this scion onto a young bald cypress seedling. the graft grew, developing into a beautiful tree with a single leader and a medium-oval crown. After seeing how well this tree developed, I propagated more, and began taking a critical look at this tree. I have been looking at seedling populations of Taxodium ascendens for nearly forty years, and this pond cypress was among the best I had ever observed—including the ‘Prairie Sentinel’ Pond Cypress, which I introduced under patent in 1974.

The ‘Morris’ pond cypress cultivar is very hardy. It has withstood temperatures of −28° F. with little or no damage.

The ‘Morris’ cultivar grows with a straight central leader, develops a medium-oval crown, and has thread-like leaves containing short scales that display a beautiful coppery-bronze autumn color. The tree has never suffered damage from high winds and has never broken from ice in the thirty-plus years that it has been under evaluation.

The following characteristics in combination distinguish the new cultivar named ‘Morris’ from other cultivars of Taxodium ascendens. To the knowledge of this inventor, there is only one other cultivar of pond cypress: The ‘Prairie Sentinel,’ which I introduced in 1973. The ‘Morris’ cultivar is medium-oval in form, vigorous in growth, with medium-green foliage. It is hardy as far north as Peoria, Ill. The ‘Prairie Sentinel’ pond cypress is very narrow in form, with only a ten foot limb spread, grows more slowly, and is not hardy north of Interstate 72. In bad winters during the 1970s, many of the the ‘Prairie Sentinel’ cypress were winter killed.

### PROPAGATION

Asexual propagation is done by bud grafting (chip budding) onto one-year-old Taxodium distichum seedlings. This method of propagation has yielded a 95-98% bud stand. Propagation is being done at Heritage Trees, Inc. near Jacksonville, Ill., and at one wholesale nursery in Oregon. Asexual propagation by this method has proven to be very consistent and effective.

### DETAILED BOTANICAL DESCRIPTION

In the following description, color references are made to The Royal Horticultural Society Colour Chart. The taxonomic description has been prepared by Thomas L. Green, Ph.D., Urban Forestry Professor, Western Illinois University, Macomb, Ill. 61455.

### BRIEF DESCRIPTION OF THE DRAWINGS

The accompanying photographs illustrate the appearance and color of the new pond cypress tree, showing the colors as accurately as is reasonably possible to obtain in colored reproductions of this type. Actual foliage colors may differ slightly due to light reflection.

FIG. 1—depicts the initially discovered tree named ‘Morris’ in summer foliage.

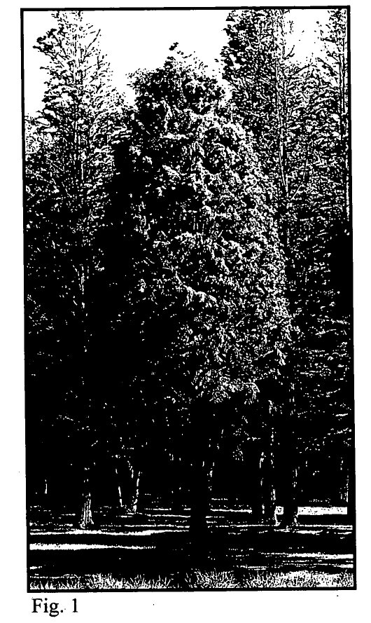

FIG. 2—depicts the awl-like foliage and the male catkins as they appear in spring.

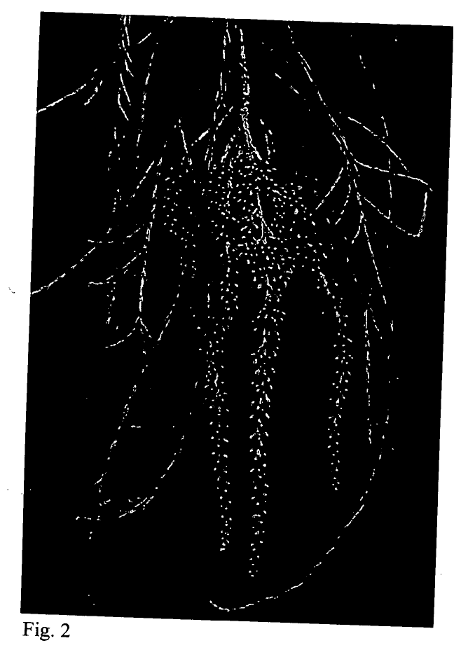

FIG. 3—depicts the seed cone as it appears in autumn.

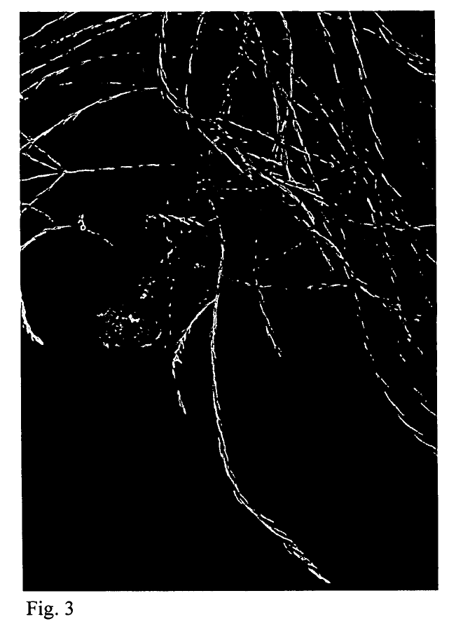

FIG. 4—depicts the tree in autumn color.

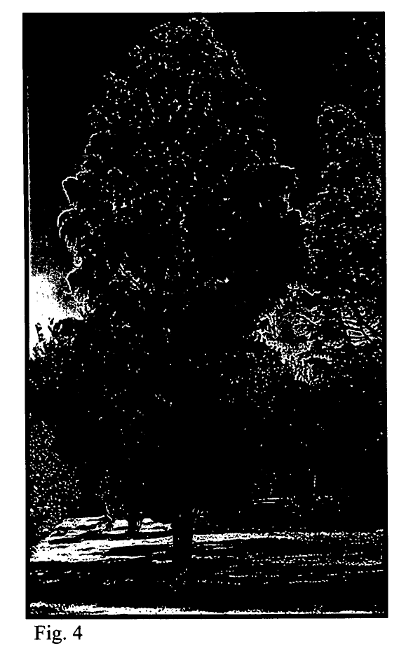

FIG. 5—depicts the tree in very early spring before foliage emerges.

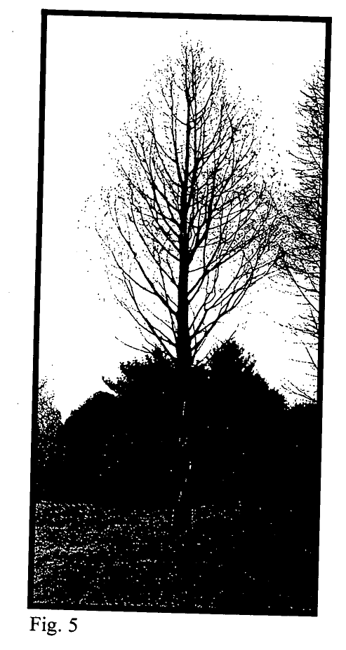

### THE PLANT

Form: Tree.
Shape: Excurrent, pyramidal.
Height: 13.1 m (40′).
Spread: 7.5 m (23′).
Bark: Trunk base flared; reddish brown (197A, 197B & 197C, with a slight tinge of 174A); long longitudinal fibrous or scaly ridges deeply and irregularly furrowed, slightly peeling.
Branches:
   Angle of attachment.—Ranges from 30° to 45°.
   Spacing.—Regular.
   Color.—Branch color changes with size Size: >1 cm (½″) (new growth) Bark: Hairless, tan (197B & 197C) Size: <1 cm (½″) Bark: Hairless, gray (199B, 197B & 197C).
Leaves:
   Scale length.—0.4-0.9 cm, μ=0.7 cm.
   Scale width.—0.1 cm, μ=0.1 cm.
   Leaf length.—2.3-12.0 cm, μ=8.9 cm.
   Leaf width.—about 1 mm.
   Form.—long, slender, flexible with numerous short, thin, flexible scales.
   Texture.—fine.
   Quantity.—abundant.
   Color.—green (139B, 139C, & 143B).
   Thorns.—none.
   Spines.—none.
   Prickles.—none.
Buds: (vegetative) tiny, <1 mm, slightly sunken.
Flowers: male: terminal. Droop in branched spikose panicle 3.0-10.0 cm (μ=6.8 cm) long.
Fruit: sessile or nearly sessile, globose 1.6-2.5 cm (μ=2.2 cm) diameter, grooved with fan-shaped and rhomboid scales; green ripening to brown

HAS THIS PLANT EVER BEEN OFFERED FOR SALE?
Yes. It was offered for sale by Heritage Seedlings, Inc. for spring 2003 delivery.

NAME THE VARIETY THIS NEW PLANT MOST RESEMBLES:
None that the inventor is aware of.

COMPARE THIS NEW VARIETY WITH OTHERS OF THE SAME SPECIES AND WITH ITS PARENT OR PARENTS; STATE THE DISTINGUISHING CHARACTERISTICS OF THE NEW VARIETY AND ITS ADVANTAGES.
The ‘Morris’ cultivar is much hardier than the species. It is a more vigorous grower. In thirty-plus years of testing, it has not suffered any breakage from wind or ice. In the inventor's opinion, it has a more pleasing autumn color than the species. The ‘Morris’ cultivar has unlimited use for street and lawn planting throughout a large portion of the United States as well as other parts of the world.

1. A new and distinct cultivar of Pond Cypress Taxodium ascendens named ‘Morris’ as illustrated and described.
